(19) United States
(12) Patent Application Publication
(10) Pub. No.: US 2004/0248182 A1
(43) Pub. Date: Dec. 9, 2004

(54) Efficient methods for multibody simulations

(75) Inventors: Dan E. Rosenthal, Los Altos, CA (US); Sia Meshkat, San Jose, CA (US)

Correspondence Address: RITTER, LANG & KAPLAN, 12930 SARATOGA AE. SUITE D1, SARATOGA, CA, 95070, US

(73) Assignee: PROTEIN MECHANICS, INC., MOUNTAIN VIEW, CA

(21) Appl. No.: 10/863,984

(22) Filed: Jun. 9, 2004

Related U.S. Application Data
(60) Provisional application No. 60/477,237, filed on Jun. 9, 2003.
(60) Provisional application No. 60/552,222, filed on Mar. 10, 2004.

(51) Int. Cl. C12Q 1/68; G06F 19/00; G01N 33/48; G01N 33/50

(52) U.S. Cl. 435/6; 702/20

(57) ABSTRACT

A fast and generally-applicable method to calculate an internal coordinate Jacobian at quadratic (order N² where N is the number of internal coordinates) cost, which can be used to dramatically speed up molecular modeling methods. In one embodiment, the present invention provides methods and algorithms useful for converting a Cartesian Hessian into a Torsion Jacobian without limitation to pair-potential energy terms, and for performing such calculation with highly-efficient usage of computer memory. Such methods and algorithms can do the conversion in computation time quadratic in the number of internal coordinates used to model any multibody system, e.g., a molecular system. In a related embodiment, the present invention provides methods and algorithms useful for computing the stiffness matrix without limitation to pair-potential energy terms, and for performing such calculation with highly-efficient usage of computer memory.

### CROSS-REFERENCES TO RELATED APPLICATIONS

This application is entitled to the benefit of the priority filing date of Provisional Patent Application No. 60/477,237, by Dan Rosenthal, filed 9 Jun. 2003; and Provisional Patent Application No. 60/552,222, by Dan Rosenthal, filed 10 Mar. 2004; both of which are hereby incorporated by reference in their entirety.

### BACKGROUND OF THE INVENTION

The present invention is related to the field of numerical methods and, more particularly, to numerical methods used in connection with solving equations of motion for mechanical systems, e.g., multibody mechanical system, particularly mechanical systems used in molecular modeling applications. All of the references cited herein are incorporated by reference for all purposes.

Molecular modeling includes a number of techniques which can be used to simulate various aspects of molecules, including their conformations, dynamic behavior and the like. Typical molecular modeling applications have included enzyme-ligand docking, molecular diffusion, reaction pathways, phase transitions, and protein folding studies. Researchers in the biological sciences and the pharmaceutical, polymer, and chemical industries are starting to use such techniques to understand the nature of chemical processes in complex molecules and to design new drugs and materials accordingly. Naturally, the acceptance of these tools is based on several factors, including the accuracy of the results in representing reality, the size and complexity of the molecular systems that can be modeled, and the speed by which the solutions are obtained.

Molecular or other mechanical system simulations are generally conducted using numerical methods to solve sets of differential equations. The speed of such simulations is therefore limited in part by the speed of the numerical methods employed, and the speed of the calculations and algorithms solved using the numerical methods. The computational speed of a mechanical (e.g., molecular) simulation may be characterized in terms of the order of dependence on the number of degrees of freedom (“DOF”) in the system, where the number of DOF is generally proportional to the number of bodies (e.g., atoms) in the system. For example, the computation time of an “order (N)” algorithm scales linearly with the number of DOF in the system, whereas that of an “order (N squared)” (order (N²)) algorithm has a quadratic dependence (i.e., it increases proportionally to the square of the number of DOF), an “order (N³)” algorithm has a cubic dependence, and so-on. As the size of the system (and therefore the number of DOF) grows, algorithms with higher order (N) dependencies can rapidly become prohibitive in terms of computational cost. Accordingly, it is highly desirable to develop algorithms that have the lowest possible order (N) dependence.

Molecular simulations have generally been carried out using either Cartesian coordinates (x,y,z coordinates for each atom or body in the system, each expressed relative to an absolute reference frame) or internal coordinates (typically dihedral or torsion angle coordinates). An advantage of using internal coordinates is that the number of DOF in the system is substantially reduced (e.g., to between about ⅙ and ⅛ of that for Cartesian coordinates), at the cost of increased complexity in at least some of the algorithms used (see, e.g., Rosenthal, WO02/061662, Aug. 8, 2002, “Method for Analytical Jacobian Computation in Molecular Modeling”; or Rosenthal, US 2003/0018455 A1, Jan. 23, 2003, “Method for Analytical Jacobian Computation in Molecular Modeling”)

Two exemplary types of molecular modeling techniques are molecular dynamics (“MD”) approaches, which simulate the motion of a molecular system through time, and Monte Carlo methods, which generate different states of a molecule or molecular system by making random changes to the atoms or bodies which make up the system, and evaluate the energy of each successive state (see, e.g., Leach, A. R., Molecular Modeling—Principles and Applications 2^{nd} Ed., 2001, Dorset Press, Dorchester, Dorset).

These, as well as other molecular and mechanical modeling techniques, are often formulated so as to make use of the first (gradient, or Jacobian) and/or second (Hessian) derivative matrixes of the conformational energy functions (potential and/or kinetic energy). These derivatives can be readily computed in Cartesian coordinates, but are very difficult to calculate from first principles in internal coordinates. Accordingly, if one wishes to find the Jacobian or Hessian of the energy (E) or force (F) functions in internal coordinates, one generally needs to convert the Cartesian Jacobian or Hessian into the corresponding internal coordinate (e.g., torsion angle coordinate) Jacobian or Hessian.

Although the computational cost of a Cartesian Jacobian is as low as order (N²), generally-applicable prior art methods for converting a Cartesian Jacobian to an internal coordinate Jacobian have a cubic dependence on N (i.e., order (N³)). The conversion calculation can thus become a bottleneck when working with large systems. Several methods for speeding up Jacobian computation in the context of molecular modeling (to as low as order N²) have been described (e.g., Gibrat, 1997, “Fast Calculation of first and second derivatives of conformational energy of oligomeric proteins with respect to a set of variables mixing dihedral angles and rigid body variables”, J. Chem Phys 94:1234-1256; Noguti & Go, 1983, J. Phys. Soc. Jap. 52:3685), but they suffer from certain limitations as detailed below.

Many techniques for calculating the force or energy Jacobian employ the chain rule; for example, the force Jacobian can be expressed in internal coordinates as

$$\frac{\partial F}{\partial q} = \frac{\partial F}{\partial r} \frac{\partial r}{\partial q} ,$$

where Cartesian coordinates are represented as r and internal coordinates are represented as q.

$$\frac{\partial F}{\partial r}$$

is typically the Cartesian Hessian whenever F itself is obtained as the gradient of a potential energy; otherwise

$$\frac{\partial F}{\partial r}$$

is referred to as the Cartesian Force Jacobian.

$$\frac{\partial r}{\partial q}$$

is referred to as the torsion displacement gradient. In the “explicit matrix multiplication method” (see, e.g., Rosenthal, WO02/061662, Aug. 8, 2002, “Method for Analytical Jacobian Computation in Molecular Modeling”; or Rosenthal, US 2003/0018455 A1, Jan. 23, 2003, “Method for Analytical Jacobian Computation in Molecular Modeling”, both incorporated herein by reference for all purposes), both the Cartesian Jacobian and the displacement gradient are formed and an explicit matrix multiplication is performed. Because of the inordinate memory requirement to store the Cartesian Jacobian for large molecular systems, some special provision is generally made to partially form the Jacobian in blocks. The cost of the matrix multiplication is cubic (order (N³)) in the number of atoms. Depending on the force field used, the cost of forming the Cartesian Jacobian can be quadratic (order (N²)). The cost of forming the displacement gradient is also quadratic, so this method is eventually dominated by the cubic cost of the matrix multiply.

Methods based on numerical perturbation (see, e.g., Lyness & Moler, 1967, “Numerical differentiation of analytic functions”, SIAM J. Numer. Anal. 4:202-210) have also been described. If the simulation engine employed is able to compute atomic forces, the atomic forces can be viewed as implicit functions of the generalized coordinates: F=F(r(q)). Therefore,

$$\frac{\partial F}{\partial q}$$

can be formed numerically, using, for instance, finite differences. The overall cost of this scheme is cubic, since each column of the Jacobian requires at least one force evaluation. The accuracy of the numerical scheme may also suffer due to round off and truncation artifacts. Complexification techniques (see, e.g., Martins, et al., 2000, “An automated method for sensitivity analysis using complex variables”, AIAA 2000-0689) can be used to restore accuracy of the obtained results, but this requires rewriting the entire force field to use complex arithmetic, an impractical requirement for many applications.

Also described in the prior art are methods based on forward mode differentiation (see, e.g., Bischof, et al., 1992, “ADIFOR: Generating Derivative Codes from Fortran Programs”, Scientific Programming 1:11 -29), which arise from application of program augmentation techniques. The application of these methods can be done either with a code generation program such as Adifor (Argonne National Laboratory and Rice University), or manually. Application of such a method results in a program that can compute the matrix vector product

$$\frac{\partial F}{\partial r} z ,$$

for a passed-in data vector z. A good use of such a program is to form the displacement gradient and pass it into the forward mode derivative routine. This still results in cubic overall cost, but is about three times faster than a numerical finite difference method, and obtains full accuracy.

In the method of Gibrat (Gibrat, 1997, “Fast Calculation of first and second derivatives of conformational energy of oligomeric proteins with respect to a set of variables mixing dihedral angles and rigid body variables”, J. Chem Phys 94:1234-1256), adapted from Noguti & Go, 1983, (J. Phys. Soc. Jap. 52:3685), the Jacobian or Torsion Hessian is generated in quadratic cost, but is restricted to energy functions expressible as pair potentials. Thus, it is not applicable to simple energy functions such as the 1-4 torsion potential (because the torsion potential is expressed as an additive sum of 4-atom terms), the 1-3 bond angle pair, and other energy functions which are not expressible exclusively as pair potentials. It is also not applicable to a number of other tools used in molecular modeling not expressible exclusively as pair potentials, such as the Generalized Born Solvent Accessibility (“GBSA”) solvent model, which is expressed as a true many-body potential, and is often used in molecular simulations utilizing an implicit solvent model.

The present invention overcomes the aforementioned and other limitations in the prior art, and provides a fast and generally-applicable method to calculate an internal coordinate (e.g., torsion angle) Jacobian at quadratic (order(N²)) cost, which can, among other applications, be used to dramatically speed up molecular modeling methods.

### SUMMARY OF THE INVENTION

In one embodiment, the present invention provides methods and algorithms useful for converting a Cartesian Hessian into a Torsion Jacobian without limitation to pair-potential energy terms, and for performing such calculation with highly-efficient usage of computer memory. Such methods and algorithms can do the conversion in computation time quadratic in the number of internal coordinates used to model any multibody system, e.g., a molecular system. In a related embodiment, the present invention provides methods and algorithms useful for computing the stiffness matrix without limitation to pair-potential energy terms, and for performing such calculation with highly-efficient usage of computer memory.

### BRIEF DESCRIPTION OF THE DRAWINGS

FIG. 1 illustrates the tree structure of a multibody system.

FIG. 2 is the matrix ε_{φ} for the multibody system shown in FIG. 1.

FIG. 3A shows symbolically the interaction between two frozen bodies.

FIGS. 3B-3F illustrate steps in the calculation of interactions between the two frozen bodies of FIG. 3A.

FIG. 4 is diagram of a computer system useful for executing methods of the present invention.

### DETAILED DESCRIPTION

I. Definitions

Unless indicated otherwise, “about” means the stated value ±30%.

Unless indicated otherwise, a “body”, in the context of a component of a representation of a molecule, is defined as a unit of the representation which is treated as a single mass or geometric structure for purposes of modeling the molecule. Accordingly, a body can be a representation of an individual atom of the molecule, a collection of atoms, or other abstract system of masses. A “rigid body” is a body that is modeled as rigid (i.e., it does not deform in response to forces exerted on it).

Unless indicated otherwise, a “fast operator implementation” means a method of evaluating matrix multiplication in which elements of the product matrix are computed, used and released “on-the-fly”, without the necessity of computing and storing the entire product matrix. A fast order implementation can thus be substantially more efficient (as high as order (N²)) than standard matrix multiplication (order (N³)), both in terms of execution speed and memory storage requirements.

Unless indicated otherwise, a “joint” is a connection between two bodies. Common joint types include pin joints, slider joints, and ball joints, but many other joint types are possible, including, but not limited to, free joints, U-joints, cylindrical joints, bearing joints, and combinations of any of the foregoing. For instance, a free joint consists of three orthogonal slider joints combined with a ball joint, and has the full 6 degrees of freedom. A more detailed discussions of joint types and their uses can be found, e.g., in Rosenthal, supra.

Unless indicated otherwise, a “frozen body” at a particular joint or pivot consists of all bodies outboard of the pivot, where the bodies move together as a rigid body.

Unless indicated otherwise, a “computationally-feasible order” refers to any order in which a particular sequence of tasks can be executed without altering the ultimate result. This concept is invoked, because in some methods, the order of certain steps is not important, so long as the steps are executed and the result is substantially the same as if they were executed in the order originally presented.

Unless indicated otherwise, the term “in silico” refers to any method or process performed using a computer.

Unless indicated otherwise, a “molecule” is any microscopic structure formed of two or more atoms that are connected by chemical bonds. Non-limiting examples of molecules include proteins (e.g., antibodies, receptors, etc), peptides, lipids, nucleic acids (e.g. natural or synthetic DNA, RNA, gDNA, cDNA, mRNA, tRNA, etc.), lectins, sugars (e.g. forming a lectin/sugar complex), glycoproteins, small molecules, organic compounds, monatomic or polyatomic structures such as salts, metals, etc.

Unless indicated otherwise, a “representation”, when applied to a molecule or molecular structure, refers to an abstract description of the molecule or molecular structure for use by a machine, e.g., in a computer simulation. For example, one representation of a molecule is a set of coordinates which collectively defines the positions of atoms or bodies, or some abstract proxy thereof, constituting the molecule. Non-limiting examples of representations of a molecule include X-ray coordinates, “pdb” (Protein Data Bank) files, and the like.

Unless indicated otherwise, the term “significant”, when used with reference to “significantly different”, “significantly inhibits” or “significantly stimulates”, refers to a difference in a quantifiable parameter between the two groups being compared that is statistically-significant using standard statistical tests.

Unless indicated otherwise, a “solvent” refers to any medium which can contain a solute molecule. Non-limiting examples of a solvent include water & other aqueous solvents, as well as organic solvents (e.g., DMSO, lipids, alcohol, etc.). The solvent may be uniform or non-uniform, and may be in solid, liquid or gaseous form.

Unless indicated otherwise, a “target molecule” is the primary molecule that is the subject of a molecular simulation. A simulation may have more than one target molecule, e.g., in simulations of 2 or more proteins interacting with one another.

Unless indicated otherwise, a “universally-applicable method” is any method suitable for use with a molecular simulation that is not limited to use with pair potentials.

II. Multi-Body Systems

In one embodiment, methods of the present invention are used in connection with a multibody system (MBS) implementation of a mechanical system (e.g., an MBS molecular simulation; see, e.g., Rosenthal, supra). The basic abstraction of MBS is that of a collection of joint-connected rigid bodies. One of the bodies, called the base, has special status in that its kinematics is referenced directly to ground. The system graph may contain loops. A loop-breaking algorithm reduces a cyclic graph to a tree, plus a set of cuts. The cuts can occur at joints or bodies. This places all joints in the tree system. A body that was cut by the loop breaking algorithm is recovered by imposing a weld joint. This joint is itself decomposed into a set of six distance constraints. The weld joint reassembles the two pieces of the original body.

An important property of a tree is that the path from any body to any other body is unique. The bodies in the tree are n in number, with the base body typically assigned the label 1. This means that the body labels do not decrease on any path from the base body to any leaf body. A leaf body is one that is connected to a single body. A regular labeling can be achieved by assigning the label n to one of the leaf bodies (there must be at least one). If this body is removed from the graph, there remains a tree with n−1 bodies. Assign the label n−1 to one of its leaf bodies, and repeat the process until all the bodies have been labeled. The details of MBS implementations of general mechanical systems have been described previously (see, e.g., Rodriguez and Kreutz in Recursive Mass Matrix Factorization and Inversion, JPL Publication 88-11, 1988). The details of MBS implementations of a molecular system, as well as relevant notation, have also been described (see, e.g., Rosenthal, supra).

III. Computation of the Residual

In an exemplary embodiment, methods of the invention are applied to a simulation of a system (e.g., a molecular system) represented as a multi-body system having equations of motion in the form M\dot{u}=ρ. The right hand side of this equation is ρ, the residual, and contains contributions from inertial forces, and active forces from the force field. The Jacobian of the residual is

$$\frac{\partial P}{\partial q} .$$

Methods of the invention are applicable to calculating contributions to the residual from the force field representing the active force component. A multibody system embodies a collection of data processing methods that can trigger computation of atomic forces and gather them into elements of the residual vector. These data processing steps can have a high level representation in terms of certain matrix operations, but in fact the actual implementation of these operations is typically in terms of algorithms whose run time scales linearly with problem size. These are the so-called O(n) methods, and are taught in the prior art (see, e.g., Rosenthal, supra). According to one aspect of the present invention, however, these O(n) methods can be applied to the Jacobian formation step:

For example, the residual is formed from the operator equation

ρ=HΦPF (1)

F is typically a 3nₐ by 1 vector. Each entry preferably consists of the global Cartesian components of the total atomic force exerted on a given atom. Matrix operators H, Φ, and P are described by Rodriguez and Kreutz in Recursive Mass Matrix Factorization and Inversion, JPL Publication 88-11, 1988. The force distribution matrix P generates multibody spatial loads acting at the pivots of the multibody system from atomic forces. Thus, one can write:

T=PF (2)

where T is typically a 6n_{b} by 1 vector. A given force acting upon a particular atom is mapped to a force and torque acting on the pivot point of the body upon which the atom resides. Thus, a given row of the matrix P corresponds to the spatial load acting on a particular body. The given row has nonzero blocks only for entries corresponding to atoms residing on the particular body. In many multibody formulations of molecular systems, each body typically contains or represents about three atoms. Accordingly, the matrix P is large and sparse, and it does not need to be explicitly formed (see, e.g., Rosenthal, supra). Rather, an algorithm that computes the matrix vector product PF may be used. The runtime of such a computation is linear in the number of atoms (Rosenthal, supra).

It is common in multibody dynamics that the transpose of an operator is also a useful quantity. In this case the operator P^{T} propagates spatial velocity (linear and angular ) from each body pivot to the atom stations located on the body. An exemplary use of this matrix is for the purpose of computing differential spatial displacements of the atoms in a molecular system.

The matrix Φ is known as the multibody transition operator. This matrix, acting upon a data vector, has the effect of shifting and accumulating the elements of the data vector from the leaf bodies of the tree down to the base body. For instance, the product ΦT generates R, a 6n_{b} by 1 vector of reaction loads. For a given body, the reaction load element represents the resultant force and torque about the body inboard pivot of those forces acting on bodies kinematically affected by motion of the body in question about its joint. The evaluation of ΦT is linear in the number of bodies.

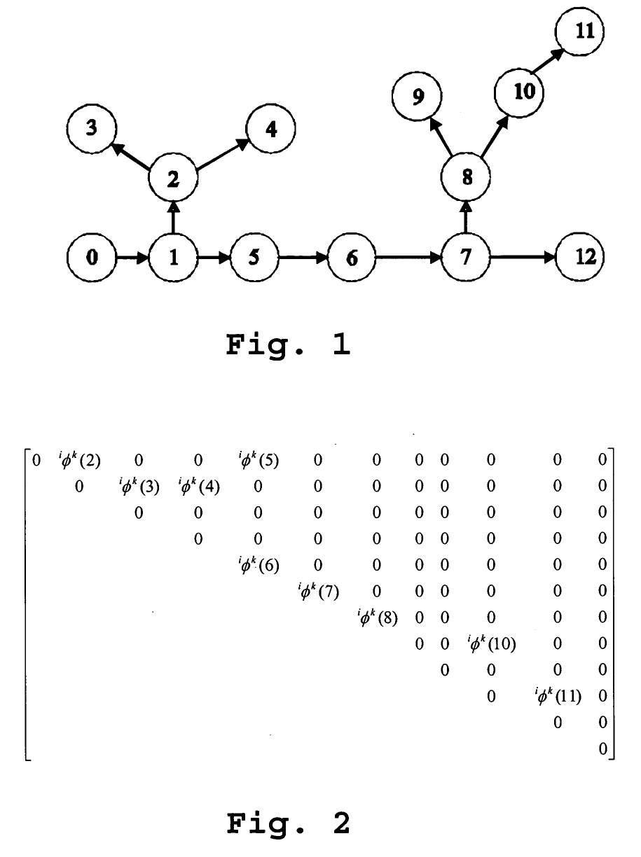

To understand Φ, it is useful to first introduce the matrix ε_{φ}. This (block) matrix is sparse and super-diagonal. In the ith row, it is filled in only in those columns corresponding to children of body i. For the body diagram shown in FIG. 1, the matrix ε_{φ} is shown in FIG. 2. Each non-zero element of ε_{φ} a 6×6 matrix. The elements ⁱφ^{k} shift spatial loads from the pivot of a child to the pivot of its parent. The transposed elements shift spatial velocity from a parent pivot to the child pivot.

Now, the relationship between Φ and ε_{φ} can be stated:

Φ=(I−ε_{φ})⁻¹ (3)

The meaning of this equation may be better appreciated upon reference to the following illustration, which describes the computation of the spatial reaction load at each pivot, given the spatial load applied to each pivot, with reference to the system shown in FIG. 1. The reaction loads can be computed by the following O(n) sweep (code sequence):

R(12)=T(12)

R(11)=T(11)

R(10)=ⁱφ^{k}(11)R(11)+T(10)

R(9)=T(9)

R(8)=ⁱφ^{k}(10)R(10)+ⁱφ^{k}(9)R(9)+T(8)

R(7)=ⁱφ^{k}(8)R(8)+ⁱφ^{k}(12)R(12)+T(7)

R(6)=ⁱφ^{k}(7)R(7)+T(6)

R(5)=ⁱφ^{k}(6)R(6)+T(5)

R(4)=T(4)

R(3)=T(3)

R(2)=ⁱφ^{k}(3)R(3)+ⁱφ^{k}(4)R(4)+T(3)

R(1)=ⁱφ^{k}(2)R(2)+ⁱφ^{k}(5)R(5)+T(1)

R(0)=ⁱφ^{k}(1)R(1)+T(0)

These equations accumulate the reaction at each pivot. The sweep starts at the leaf bodies and traverses the tree to the base body. Each body shifts its reaction to its parent body, where it is added to the contribution from its brothers. Notice that the equations make backwards references to previously computed quantities. For instance, R(6) refers to R(7), which refers to R(8) and R(12), etc. However, if the algorithm is arranged as presented, no unsatisfied references are encountered. The correspondence between the operator equation R=ΦT and the above sequence can be illustrated as follows—the sequence shows the equation R=ε_{φ}R+T, from which (I−ε_{φ})R=T, or R=(I−ε_{φ})⁻¹T=ΦT. To go the other direction (from equation to code sequence), the steps can simply be reversed:

$$\begin{matrix}R = Φ T \\ = {( I - {ϵ}_{φ} )}^{- 1} T , \text{or} \\ ( I - {ϵ}_{φ} ) R = T , \text{from which} \\ R = {ϵ}_{φ} R + T\end{matrix} \qquad ( 4 )$$

If one wishes to expose the elements of Φ, one can, for example, eliminate the backwards references in the computation of R by performing repeated substitutions, and then identify the matrix elements of Φ. Alternatively, one may employ an algorithmic alternative. Since (I−ε_{φ})Φ=I, we have Φ=ε_{φ}Φ+I, and this equation has the same structure as R=ε_{φ}R+T. The same algorithm can be applied to the columns of Φ, starting from the last column and working backwards to the first. However, as will be evident, there is typically no circumstance in which the elements of Φ are needed, since only the action of the operator is needed. Therefore, all algorithms which indicate multiplication by Φ (a dense upper triangular matrix) may proceed in terms of ε_{φ}, a sparse super-diagonal matrix. To finish the residual computation, the matrix vector product HR projects the elements of the vector R onto the joint freedoms. The matrix H is a block diagonal. Each block is filled with the joint map for a particular joint. The map for a pin joint is a 1×6 vector [λ 0], where the unit vector λ specifies the pin geometry. The matrix vector product HR is easily computed in linear cost, since it represents a sequence of non-recursive dot-products. The operator H has the following shape, where n is the number of bodies and nᵤ is the number of generalized speeds:

$$H = \begin{matrix}\begin{matrix}1 \\ \end{matrix} \\ n\end{matrix} {[ \begin{matrix}[ {n}_{{u}_{1}} \times 6 ] & ⋯ & [ {n}_{{u}_{1}} \times 6 ] \\ ⋯ & ⋯ & ⋯ \\ [ {n}_{{u}_{n}} \times 6 ] & ⋯ & [ {n}_{{u}_{n}} \times 6 ]\end{matrix} ]}^{\begin{matrix}1 & & n\end{matrix}} = \begin{matrix}\begin{matrix}1 \\ \end{matrix} \\ {n}_{u}\end{matrix} {[ \begin{matrix}[ 1 \times 6 ] & ⋯ & [ 1 \times 6 ] \\ ⋯ & ⋯ & ⋯ \\ [ 1 \times 6 ] & ⋯ & [ 1 \times 6 ]\end{matrix} ]}^{\begin{matrix}1 & & n\end{matrix}}$$

There is a corresponding operator \tilde{H} which has the following shape, where n_{q} is the number of generalized coordinates:

$$\tilde{H} = \begin{matrix}\begin{matrix}1 \\ \end{matrix} \\ n\end{matrix} {[ \begin{matrix}[ {n}_{{q}_{1}} \times 6 ] & ⋯ & [ {n}_{{q}_{1}} \times 6 ] \\ ⋯ & ⋯ & ⋯ \\ [ {n}_{{q}_{n}} \times 6 ] & ⋯ & [ {n}_{{q}_{n}} \times 6 ]\end{matrix} ]}^{\begin{matrix}1 & & n\end{matrix}} = \begin{matrix}\begin{matrix}1 \\ \end{matrix} \\ {n}_{q}\end{matrix} {[ \begin{matrix}[ 1 \times 6 ] & ⋯ & [ 1 \times 6 ] \\ ⋯ & ⋯ & ⋯ \\ [ 1 \times 6 ] & ⋯ & [ 1 \times 6 ]\end{matrix} ]}^{\begin{matrix}1 & & n\end{matrix}}$$

In view of the foregoing, it can be appreciated that the computation of the residual can be seen as a sequence of O(n) methods applied to the elements of the atomic forces computed by the force field. These include, for example, vacuum terms, solvent terms (polar and non-polar), and velocity related terms. Several of these items require quadratic compute time. For example, in many implementations, electrostatics and the GBSA solvent model generally consume the majority of the computational time for force evaluation. Thus, the residual cost is quadratic in the number of atoms. This means that the Jacobian of the residual is of quadratic cost at best, and possibly worse.

Given that ρ=HΦPF,

$$\frac{\partial ρ}{\partial q}$$

can be expressed as:

$$\frac{\partial ρ}{\partial q} = H Φ P \frac{\partial F}{\partial q} + \frac{\partial ( H Φ PF )}{\partial q} \qquad ( 5 )$$

The atomic force vector F appears in both the first and second terms. However, the second term only requires the numeric values of the force vector, not knowledge of its analytic form. Thus, this term represents the derivative of a term whose original cost is O(n), because no new force evaluations are needed during evaluation of this term. One can thus demonstrate that this term can only give rise to quadratic cost in the Jacobian. This can be appreciated by noting that that the residual computation can be differentiated using forward mode differentiation, treating the force vector as a known constant, thereby resulting in a program whose cost is bounded by a constant times the original program cost (per call), as described in, e.g., Bischof, et al., 1994, (“Automatic differentiation: obtaining fast and reliable derivatives—fast” Proc. SLAM Symposium on Control Problems in Industry Argonne, Preprint MCS-P484-1194). The constant depends on the nature of the intrinsic functions appearing in the program. The multibody system contains only trigonometric, single argument functions, in addition to arithmetic operations. Thus, in general, the constant is typically on the order 1˜2 times the original program cost or less, and the second term may be easily computed in at most quadratic cost for the whole Jacobian. Computation of the first term in the Jacobian (which involves

$$( which involves \frac{\partial F}{\partial q} )$$

) is described below.

IV. Computation of

$$\frac{\partial F}{\partial q}$$

According to an aspect of the present invention, the displacement gradient may be expressed in terms of O(n) multibody operators P, Φ, H:

$$\frac{\partial r}{\partial q} = {P}^{T} {Φ}^{T} {\tilde{H}}^{T} \qquad ( 6 )$$

In one embodiment, particularly applicable to multibody system implementations, these operators are used to form the displacement gradient in quadratic cost. Without the use of these operators, the cost would be cubic. This approach makes use of the fact that the displacement gradient is not needed explicitly, only the matrix product with the Cartesian Jacobian is needed. The product can be formed in terms of O(n) operators since

$$\frac{\partial F}{\partial q} = \frac{\partial F}{\partial r} \frac{\partial r}{\partial q} = {( \tilde{H } Φ P \frac{\partial {F}^{T}}{\partial r} )}^{T} \qquad ( 7 )$$

This is the same operator discussed above in connection with computation of the residual. Thus, this method yields the internal coordinate (e.g., torsion) Jacobian in linear cost per column, or quadratic cost overall, provided the Cartesian Jacobian is computable in quadratic cost.

The above relations may be combined to yield

$$\begin{matrix}H Φ P \frac{\partial F}{\partial r} {P}^{T} {Φ}^{T} {\tilde{H}}^{T} = \\ H Φ ( P \frac{\partial F}{\partial r} {P}^{T} ) {Φ}^{T} {\tilde{H}}^{T} = \\ H Φ W {Φ}^{T} {\tilde{H}}^{T} = \\ H {Φ ( \tilde{H} Φ W )}^{T}\end{matrix} \qquad ( 8 )$$

where the matrix

$$W = ( P \frac{\partial F}{\partial r} {P}^{T} ) .$$

In cases where matrix W is formed in a multibody system, Wᵢⱼ, a generic element of the matrix, reflects the disposition of the bodies of the multibody system in space. The element is used to compute the derivative of spatial load acting on body i due to a spatial displacement of the pivot of body j. The computation of the elements of W is where the connection to the Cartesian Hessian is made, and we have the equation:

$${W}_{ij} = {\sum }_{k ∈ i} {\sum }_{l ∈ j} {P}_{ik} \frac{\partial {F}_{k}}{\partial {r}_{l}} {P}_{jl}^{T} . \qquad ( 9 )$$

Some features of this approach bear pointing out: (i) this equation uses Hessian elements. It is guided by the atom list of body i against the atom list of body j; (ii) since each body typically consists of roughly three atoms, this is a simple computation; (iii) since each atom belongs to only one body, each element of the Cartesian Hessian will contribute to only one element Wᵢⱼ; and unlike prior art methods (e.g., Gibrat, J. F. (1997) “Fast calculation of the first and second derivatives of the conformational energy of oligomeric proteins with respect to a set of variables mixing dihedral angles and rigid-body variables”, J Chem Phys 94, 1234-1256), the method described above uses Hessian elements directly. Therefore, there is no restriction to pair potentials. The corresponding quantity Lᵢⱼ in the paper by Gibrat (supra) is built directly from the atoms of body i against the atoms of body j, but in general, Lᵢⱼ≠Wᵢⱼ. The use of Hessian elements as described herein leads to a simpler recursion sequence.

In a fast operator implementation, the overall computation represents two composed down-loops. The term \tilde{H}ΦW is computed by the following recursive algorithm. First, let Y=ΦW, Z=HY. This yields

(I−ε_{φ})Y=W, or

Y=ε_{φ}Y+W (10)

As described above, the matrix ε_{φ} is subdiagonal and is populated with the elements ⁱφ^{k}(k). One may then find the ‘stacked’ equations

Y₁=W₁

Y₂=¹φ²Y₁+W₂

Y₃=²φ³Y₂+W₃ (11)

In these equations W₁ represents the first (logical) row and Wₙ represents the last (logical) row of W. This is a 6 by 6n_{b} block. Each row of W is computed in terms of prior rows. The computation Z=HY is trivial, since H has a block-diagonal structure.

The second recursion can now be expressed as follows:

$$\frac{\partial ρ}{\partial q} = H Φ {Z}^{T} \qquad ( 12 )$$

The operator HΦ can be seen to be operating on the rows of Z. This dovetails nicely with the first recursion, which generated Z in row order. The second recursion only needs to compute elements in a given column up to the diagonal, since the destination matrix is symmetric.

In view of the foregoing, it can be appreciated that the atomic force Jacobian can be contracted to form W. The overall algorithm only requires row (or column access) to the Force Jacobian, and it is not necessary to access the entire matrix at once. It will also be appreciated that the first and second recursions can be interspersed if desired. In this way an element of Y or Z can be released as soon as it has been computed. It is thus not necessary to form all of Y or Z in one step.

V. Memory-Efficient Computation

The above-described method may be implemented in a manner that minimizes the amount of memory necessary to perform the calculations, by more fully exploiting the symmetry of W. One approach to decreasing memory requirements is to use a ‘slicing’ technique that produces the Cartesian Hessian in large blocks, and then processing each block. Such a method would be viable, in the sense that it would decrease memory usage, but execution time might suffer. Methods described hereunder were designed to provide a processing scheme that could substantially reduce memory requirements without sacrificing execution speed.

In one embodiment, the method is based on expressing the Torsion Jacobian as

$$H Φ P \frac{\partial F}{\partial r} {P}^{T} {Φ}^{T} {\tilde{H}}^{T} = \\ H Φ ( P \frac{\partial F}{\partial r} {P}^{T} ) {Φ}^{T} {\tilde{H}}^{T} = \\ H ( Φ W {Φ}^{T} ) {\tilde{H}}^{T} = \\ H Ω {\tilde{H}}^{T} \qquad ( 13 )$$

The new matrix Ω bears the same relationship to W that the reaction R bears to the applied load T. The matrix W is a spatial Hessian for the bodies of a multibody system. It is the Hessian that would be relevant for the bodies floating in space, but not connected by joints. Elements of the matrix Ω couple ‘nests’ of frozen bodies. At each pivot, the frozen body is composed of all bodies outboard of the pivot, the bodies moving together as a rigid body.

A differential spatial displacement at the pivot of each frozen body causes a differential spatial load at the pivot of all the other frozen bodies. Considering a displaced body as the ‘source’ body, and a body experiencing the load as the ‘receiver’, it can be appreciated that each source body interacts with each receiver body. However, the spatial moves in a source body originate from the pivot of the frozen body and propagate out through the action of Φ^{T} to the other bodies in the source system. Each source body produces a differential spatial load on each body of the receiver system through the coupling matrix W. The spatial loads are given by Wφ^{T}. The spatial loads of the receiver system are aggregated to the receiver pivot through the action of the operator Φ acting on (WΦ^{T}).

In a fast operator implementation, Ω can be efficiently computed as follows:

Ω=ΦWΦ^{T}, or

(I−ε_{φ})Ω(I−ε_{φ}^{T})=W, from which

Ω=ε_{φ}Ω+Ωε_{φ}^{T}−ε_{φ}Ωε_{φ}^{T}+W (14)

The final equation provides a basis for computing Ω. Since ε_{φ} is super diagonal (nonzero only above the main diagonal), the term ε_{φ}Ω couples low numbered rows of Ω to higher numbered rows, but not vice versa. That is, in a given column elements move up the column when Ω is pre-multiplied by ε_{φ}. Similarly, the term Ωε_{φ}^{T} only couples low numbered columns to higher numbered columns. Elements move left across a given row. The third term couples both early rows and early columns to higher rows and higher columns, but coupling does not occur in the other direction. Elements move either up, left, or up and to the left. The net result is that Ω can always be evaluated in reverse column order, as long as the tree system is regularly labeled.

A desirable labeling is one that minimizes the bandwidth of ε_{φ}. The bandwidth of ε_{φ} limits the ‘reach’ of the matrix and determines how far ‘back’ the backwards references can extend during the computation of Ω.

By way of example, the above algorithm can be applied to a subset of the system shown in FIG. 1 by evaluating the following equations:

Ω(12,12)=W(12,12)

Ω(12,11)=W(12,11)

Ω(12,10)=W(12,10)+Ω(12,11)ⁱφ^{k*}(11)

Ω(12,9)=W(12,9)

Ω(12,8)=W(12,8)+Ω(12,9)ⁱφ^{k*}(9)+Ω(12,10)ⁱφ^{k*}(10)

Ω(12,7)=W(12,7)+Ω(12,8)ⁱφ^{k*}(8)+Ω(12,12)ⁱφ^{k*}(12)

The first two equations pick up only the direct contribution of the bodies through the coupling matrix W. The (12,10) element computes the spatial load on frozen body 12 due to spatial displacement of frozen body 10. Frozen body 10 consists of actual bodies 10 and 11. The displacement at the pivot of 10 propagates to 11 through ⁱφ^{k*}(11) . This displacement produces a spatial load on 12 through the coupling element Ω(12,11). The (12,7) element is slightly more complicated. Frozen body 7 consists of body 7, plus frozen bodies 8 and 12. These are frozen bodies corresponding to the children of body 7. A displacement at the pivot of body 7 propagates out to its children, couples to the already computed elements of Ω, and generates a load on the pivot of body 12.

Without recursion, it would be necessary to compute over the nest of bodies comprising the frozen bodies of the children. This would become an ever growing list as the computation progressed. With recursion, it is necessary to only visit frozen bodies of children. Also, since each body actually has a small number of children (usually 2 or 3, but in some cases more) the cost per element is relatively small.

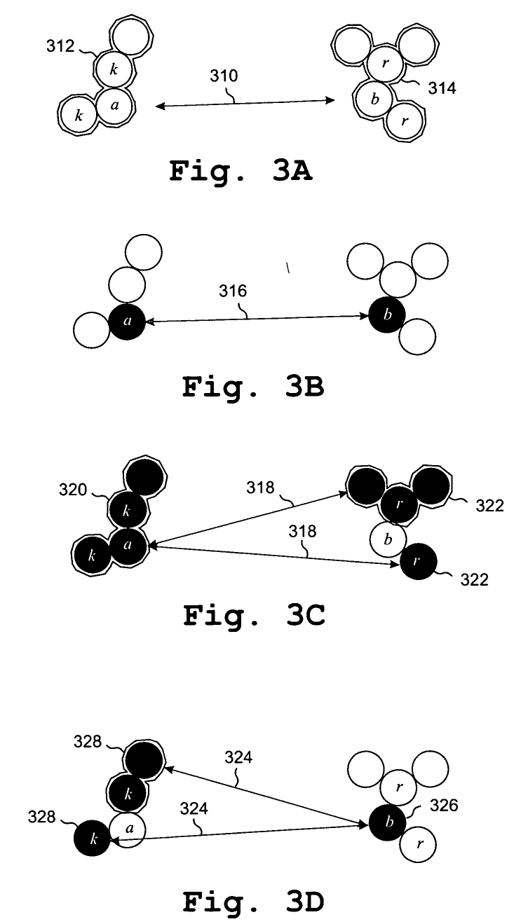

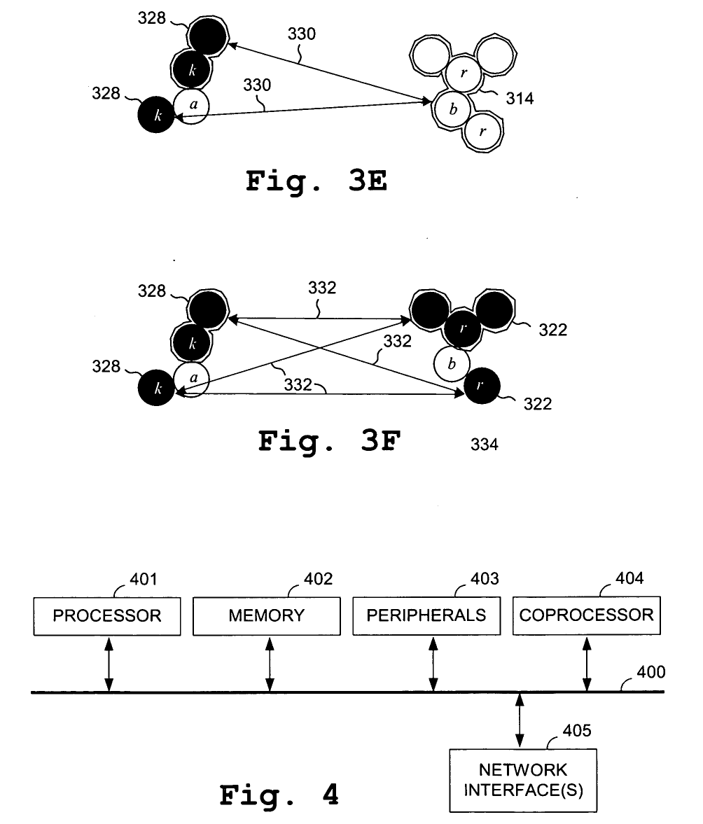

The approach described below and illustrated in FIGS. 3A-3F shows a calculation of the interactions between two frozen bodies. The operations described are in fact performed “automatically”, in the course of the computing the equations described above, and are shown to allow better appreciation and implementation of the methods. To facilitate keeping track of bodies under consideration and their children, a table, such as Table 1 below, may be constructed.

TABLE 1

| Body a | Body b |
|---|---|
| k ∈ children(a) | r ∈ children(b) |

Turning now to FIG. 3A, the drawing shows an interaction 310 between frozen body 312 and frozen body 314. In this example, a≧b; frozen body 312 (the “receiving body”) contains body a, its children k, and one grandchild of a; and frozen body 314 (the “sourcing body”) contains body b, its children r, and two grandchildren of b. Interaction 310 may be determined by decomposition via the steps shown in FIGS. 3B-3D, performed in any computationally-feasible order: For example, first, direct term 316 between the two individual bodies a and b as shown in FIG. 3B is determined. Term 318 (obtained from Ωε_{φ}^{T}), produced by frozen body a 320 interacting with the frozen children of b 322 as shown in FIG. 3C, is then added. The final interaction needed (324) is that between body b 326 against k (328), the frozen children of a, as shown in FIG. 3D. This term is preferably computed indirectly, because body b by itself is not a frozen body. In one embodiment, the interaction of body b against the frozen children of a is computed by subtraction: first, as illustrated in FIG. 3E, the interaction 330 between frozen body b 314 against the frozen children of a 328 is computed, giving rise to terms obtained from ε_{φ}Ω. Then, as shown in FIG. 3F, interaction 332 between frozen children of a against frozen children of b (term ε_{φ}Ωε_{φ}^{T}) is subtracted. This leaves the interaction b against the frozen children of a as shown in FIG. 3D.

By way of example, the above-described operations may be applied to the analysis of a portion of the multi-body system shown in FIG. 1. First, any element in which both bodies have children will be a generic case. An example of such an element is Ω(8,6):

| | |
|---|---|
| 8 | 6 |
| 9 10 | 7 |

| | |
|---|---|
| | (16) |
| $\begin{matrix}Ω ( 8 , 6 ) = W ( 8 , 6 ) + {Ω ( 8 , 7 )}^{i} {φ}^{{k}^{*}} ( 7 ) + \\ {}^{i} {φ}^{k} ( 9 ) Ω ( 9 , 6 ) {+}^{i} {φ}^{k} ( 10 ) Ω ( 10 , 6 ) - \\ {}^{i} {φ}^{k} ( 9 ) {Ω ( 9 , 7 )}^{i} {φ}^{{k}^{*}} ( 7 ) - \\ {}^{i} {φ}^{k} ( 10 ) {Ω ( 10 , 7 )}^{i} {φ}^{{k}^{*}} ( 7 )\end{matrix}$ | |

Diagonal elements are treated by the same method as non-diagonal elements. An example is given for Ω(5,5):

| | |
|---|---|
| 5 | 5 |
| 6 | 6 |

| | |
|---|---|
| | (17) |
| $\begin{matrix}Ω ( 5 , 5 ) = W ( 5 , 5 ) + {Ω ( 5 , 6 )}^{i} {φ}^{{k}^{*}} ( 6 ) + \\ {}^{i} {φ}^{k} ( 6 ) Ω ( 6 , 5 ) {-}^{i} {φ}^{k} ( 6 ) {Ω ( 6 , 6 ) }^{i} {φ}^{{k}^{*}} ( 6 )\end{matrix}$ | |

Of course, it should be recognized that Ω(5,6)=Ω(6,5)^{T} due to symmetry of the matrix. To summarize, each element is computed using the formula

$$Ω ( a , b ) = W ( a , b ) + {\sum }_{r ∈ child ( a )} {}_{}^{i}{φ}{}_{}^{k} ( r ) Ω ( r , b ) + \\ {\sum }_{s ∈ child ( b )} Ω {( a , s )}^{i} {φ}^{{k}^{*}} ( s ) - {\sum }_{{r ∈ child ( a )}_{s ∈ child ( b )}} {}_{}^{i}{φ}{}_{}^{k} ( r ) {Ω ( r , s )}^{i} {φ}^{{k}^{*}} ( s ) \qquad ( 18 )$$

Guided by the tabular construction, the matrix can be computed in reverse order. At each step, a particular element of W is accessed. This is the only point at which access to that element of W will be needed. Therefore, W is preferably computed by a method that provides element-by-element access. It is not necessary to store any elements of W. Similar considerations apply to the Cartesian Hessian, since W is built from the Cartesian Hessian.

The situation is a bit different for the matrix Ω. As soon as an element Ω_{b(i),b(j)} has been computed one can generate the (ij) element of the torsion Jacobian:

$$\frac{δ {ρ}_{i}}{δ {q}_{j}} = {H}_{i} {Ω}_{b ( i ) b ( j )} {\tilde{H}}_{j}^{T} \qquad ( 19 )$$

Note that the storage used for this element of Ω is preferably not automatically released until it is known that there will not be any backwards references to it later in the algorithm. This can be determined from the known bandwidth of ε_{φ}, which bounds the maximum reference possible. For instance, in the multibody system of FIG. 1, body 7 is the parent for body 12. This means that storing 5 columns of Ω will be enough. Similarly, the (7,2) element references elements (7,3), (7,4), (8,2), (12,2), (8,3), (8,4), (12,3), and (12,4). These elements all lie within a band five columns away from (7,2). In practice, storing a small number of columns is generally sufficient to avoid re-computation.

VII. Computer System

To carry out the calculations described above, a computer system may be used with at least one processor and associated memory subsystem for holding the computer code to instruct the processor to perform the operations described above. FIG. 4 illustrates the basic architecture of such a computer system having a processor 401, a memory subsystem 402, peripherals 403 such as input/output devices (keyboard, mouse, display, etc.), perhaps a co-processor 404 to aid in the computations, and network interface devices 405, all interconnected by a bus 400. The memory subsystem optimally includes, in increasing order of access latency, cache memory, main memory and permanent storage memory, such as hard disk drives. Given the amount of intensity of computation, it should be understood that the computer system could include multiple processors with multiple associated memory subsystems to perform the computations in parallel; or, rather than having the various computer elements connected by a bus in conventional computer architecture as illustrated by FIG. 4, the computer system might formed by multiple processors and multiple memory subsystems interconnected by a network.

The following examples illustrate but in no way are intended to limit the present invention.

### EXAMPLE 1

### Computational Speed Comparison of Previous Methods and Methods of the Present Invention

The dynamic residual ρᵤ is computed from the atomic forces F as follows:

ρᵤ=HΦPF (20)

The derivatives of ρᵤ can be obtained using previous methods by using:

$$\frac{\partial }{\partial q} {ρ}_{u} = H Φ P ( \frac{\partial }{\partial q} F ) + \frac{\partial }{\partial q} ( H Φ P ) F \qquad ( 21 )$$

which employs

$$\frac{\partial }{\partial q} F ,$$

the a mixed Cartesian-Torsion Derivative Matrix.

Using methods of the present invention, however, the dynamic residual derivative can now be computed as:

$$\frac{\partial }{\partial q} {ρ}_{u} = H Φ P ( \frac{\partial }{\partial r} F ) {P}^{T} {Φ}^{T} {\tilde{H}}^{T} + \frac{\partial }{\partial q} ( H Φ P ) F \qquad ( 22 )$$

Methods detailed herein were combined with previous approaches as follows: For convenience, the atomic forces were split up into two components:

F=F₁+F₂ (23)

in such a way that their derivatives are computed in two separate terms

$$\frac{\partial }{\partial r} {F}_{1} and \frac{\partial }{\partial q} {F}_{2} .$$

Then, the derivatives of ρᵤ were written as:

$$\frac{\partial }{\partial q} {ρ}_{u} = H Φ P ( \frac{\partial }{\partial r} {F}_{1} ) {P}^{T} {Φ}^{T} {\tilde{H}}^{T} + H Φ P ( \frac{\partial }{\partial q} {F}_{2} ) + \frac{\partial }{\partial q} ( H Φ P ) ( {F}_{1} + {F}_{2} ) \qquad ( 24 )$$

where the first is the Cartesian Hessian according to an embodiment of the present invention and the second is a mixed Cartesian-Torsion Hessian as used previously.

Basic Algorithm

The following expression was computed according to methods described herein:

$${ρ}_{u}^{′} = H Φ P ( \frac{\partial }{\partial r} {F}_{1} ) {P}^{T} {Φ}^{T} {\tilde{H}}^{T} \qquad ( 25 )$$

and added to the rest of

$$\frac{\partial }{\partial q} {ρ}_{u}$$

computed by existing methods.

This was rewritten as:

$$\begin{matrix}{ρ}_{u}^{′} = H Φ ( P ( \frac{\partial }{\partial r} {F}_{1} ) {P}^{T} ) {Φ}^{T} {\tilde{H}}^{T} \\ = H Φ W {Φ}^{T} {\tilde{H}}^{T} \\ = H {Φ ( \tilde{H} ΦW )}^{T}\end{matrix} \qquad ( 26 )$$

to allow:

1. Computation of the “body” Hessian

$$W = P ( \frac{\partial }{\partial r} F ) {P}^{T} ;$$

P^{T}; and

2. Computation of ρᵤ′, using the following sequence of operators:

a. Y=ΦW

b. Z=\tilde{H}Y

c. X=ΦZ^{T}

d. ρᵤ′=HX

The Low-Memory Algorithm

The low-memory algorithm only stores O(N) intermediate memory. The expression for the dynamic residual q-derivative can be written as follows:

$$\begin{matrix}{ρ }_{u}^{′} = H Φ ( P ( \frac{\partial }{\partial r} {F}_{1} ) {P}^{T} ) {Φ}^{T} {\tilde{H}}^{T} \\ = H Φ W {Φ}^{T} {\tilde{H}}^{T} \\ = H Ω {\tilde{H}}^{T}\end{matrix} \qquad ( 27 )$$

The matrices W and Ω can be computed in an efficient, element-by-element method:

Reorder bodies to minimize the difference m between child and parent index

Reserve memory for m columns of matrix Ω

for body i=n to 1

for body j=i to 1

| | |
|---|---|
| ${W}_{i , j} = {P}_{i} ( \frac{\partial {F}_{1}}{\partial r} ) {P}_{j}^{T}$ | (28) |
| ${Ω}_{i , \overline{j}} = {W}_{i , j} + {Φ}_{{c}_{i}} {Ω}_{{c}_{i} , \overline{j}} + {Ω}_{i , {\overline{c}}_{j}} {Φ}_{{c}_{j}}^{T} - {Φ}_{{c}_{i}} {Ω}_{{c}_{i} , {\overline{c}}_{j}} {Φ}_{{c}_{j}}^{T}$ | (29) |
| $\frac{\partial {ρ}_{u ( i )}}{\partial {q}_{q ( i )}} = {H}_{u ( i )} {Ω}_{i , j} {\tilde{H}}_{q ( j )}^{( T )}$ | (30) |

end

end

where

\tilde{k} is equal to k mod(m)

cᵢ is the set of children of body i

qᵢ is the set of q-indices for body i

uᵢ is the set of u-indices for body i

In the above, Einstein summation convention is used. The elements of matrix W are computed from

$$\frac{\partial {F}_{1}}{\partial r}$$

as needed.

### EXAMPLE 2

### Computation of the Jacobian Matrix

The Jacobian matrix consists of 4 blocks

$$J ( q , u ) = [ \begin{matrix}{J}_{qq} & {J}_{qu} \\ {J}_{uq} & {J}_{uu}\end{matrix} ] = [ \begin{matrix}\frac{\partial \dot{q}}{\partial q} & \frac{\partial \dot{q}}{\partial u} \\ \frac{\partial \dot{u}}{\partial q} & \frac{\partial \dot{u}}{\partial u}\end{matrix} ] \qquad ( 31 )$$

All but the lower left block are straight-forward to compute. According to methods described herein, the lower left block can be computed from the mass matrix M and the dynamic residual ρᵤ:

$$\dot{u} = {M}^{- 1} {ρ}_{u} \\ \frac{\partial \dot{u}}{\partial q} = \frac{\partial }{\partial q} ( {M}^{- 1} {ρ}_{u} ) = \frac{\partial }{\partial q} ( {M}^{- 1} ) {ρ}_{u} + {M}^{- 1} \frac{\partial }{\partial q} {ρ}_{u} \qquad ( 32 )$$

The last term involves the derivative of dynamic residual with respect to the generalized coordinates, as described in the present invention.

### EXAMPLE 3

### Computation of the Stiffness Matrix

Let the system possess n_{q} generalized coordinates and nᵤ generalized speeds. If there are no constraints or quaternion joints, then n_{q}=nᵤ. In this case, the full-rank (non-singular) stiffness matrix with respect to the independent set of generalized coordinates can be computed directly:

$$K = \frac{\partial }{\partial q} {ρ}_{u} \qquad ( 33 )$$

This is an nᵤ×nᵤ matrix.

However, in the presence of quaternions and/or constraints, n_{q}>nᵤ. In this case, the stiffness matrix can be computed in two steps, the first of which is a subset of the Jacobian computation:

1. The singular stiffness matrix with respect to the full set of (dependent) generalized coordinates, is computed as the derivative of the dynamic residual ρᵤ:

$$\tilde{K} = \frac{\partial }{\partial q} {ρ}_{u} \qquad ( 34 )$$

This is an n_{q}×n_{q} matrix.

2. The non-singular stiffness matrix is then computed from this matrix, by elimination of the dependent generalized coordinates. This can be done using any of a number of standard approaches. For example, one can use singular-value decomposition of the constraint matrix. If G represent the constraint matrix, one computes its null space Z of G, by solving GZ=0. Then the full-rank stiffness matrix can be computed as:

K=Z^{T}\tilde{K}Z

One application of the stiffness matrix is in the computation of normal modes, as is well-known in the art.

Results

The above implementation was used to compute

$$\frac{\partial }{\partial q} {ρ}_{u}$$

of lysozyme (±omegas) and trypsin (+omegas) using the existing methods and methods of the present invention. The results are summarized in Table 1, below.

TABLE 1

| Protein | nQ | Nu | # atoms | Speed (PA) | Speed (MI) |
|---|---|---|---|---|---|
| Lysozyme | 514 | 513 | 1666 | 43 | 14 |
| (no omegas) | | | | | |
| Lysozyme | 620 | 619 | 1666 | 77 | 18 |
| (+omegas) | | | | | |
| Trypsin | 1238 | 1238 | 3223 | 553 | 80 |
| (+omegas) | | | | | |

Speed (PA) is the computational speed (in seconds) using the previous methods described above; speed (MI) is the computational speed (in seconds) using methods of the invention. It can be appreciated that methods of the invention can substantially increase the computation speed of

$$\frac{\partial }{\partial q} {ρ}_{u}$$

relative to previously-used methods.

The Table 2 shows the results from the basic algorithm, and the low-memory (LM) implementations of the Fast Jacobian algorithm.

TABLE 2

| | | | | Memory | | | CPU Time | | | |
|---|---|---|---|---|---|---|---|---|---|---|
| | | | | Peak | | | Jacobian breakdown | | | |
| | | | | Heap | Jacobian | dRhoU/d | dRhoU/dQ breakdown | | | |
| Molecule | nRes | nAtoms | Algorithm | (MB) | Total | Q Total | dF/dR | dF/dU | dF/dQ | other |
| | | | LM | 62 | 8.5 | 7.8 | 3.3 | 0.9 | 1.6 | 2.0 |
| HIV | | | B | 96 | 9.2 | 8.4 | 3.9 | 0.9 | 1.6 | 2.0 |
| Protease | | | | | | | | | | |
| subset | 75 | 1196 | Change | 35% | 8% | | 15% | | | |
| | | | LM | 100 | 14.6 | 13.2 | 5.4 | 1.6 | 2.7 | 3.5 |
| HIV | | | B | 150 | 15.9 | 14.5 | 6.7 | 1.6 | 2.7 | 3.5 |
| Protease | | | | | | | | | | |
| monomer | 99 | 1565 | Change | 33% | 8% | | 19% | | | |
| | | | LM | 155 | 23.3 | 20.9 | 8.6 | 2.6 | 4.2 | 5.6 |
| | | | Basic | 250 | 25.4 | 23.0 | 10.7 | 2.6 | 4.2 | 5.6 |
| Lysozyme | 129 | 1969 | Change | 38% | 8% | | 20% | | | |
| | | | LM | 196 | 31.6 | 28.2 | 11.9 | 3.4 | 5.6 | 7.3 |
| | | | Basic | 320 | 34.2 | 30.7 | 14.4 | 3.4 | 5.6 | 7.3 |
| Calmodul- | 144 | 2209 | Change | 39% | 8% | | 17% | | | |
| in | | | LM | 380 | 63.3 | 56.4 | 24.2 | 7.0 | 11.3 | 13.9 |
| HIV | | | Basic | 626 | 69.5 | 62.6 | 30.3 | 7.0 | 11.4 | 13.9 |
| Protease | | | | | | | | | | |
| dimer | 198 | 3130 | Change | 39% | 9% | | 20% | | | |
| | | | LM | 405 | 69.2 | 61.4 | 26.4 | 7.3 | 12.2 | 15.5 |
| | | | Basic | 724 | 74.2 | 66.1 | 31.2 | 7.3 | 12.2 | 15.4 |
| Trypsin | 223 | 3223 | Change | 44% | 7% | | 15% | | | |
| P38 subset | | | LM | 867 | 163.3 | 145.3 | 64.7 | 16.3 | 27.1 | 37.2 |
| (300 | | | Basic | N/A | | | | | | |
| residues) | 300 | 4851 | Change | | | | | | | |

Scalability

The Cartesian Hessian matrix

$$\frac{\partial }{\partial r} F$$

can be large, and may eventually exceed the 32-bit address space. To resolve this issue, the matrix can be constructed in parts. The algorithm can be made scalable by operating on a row of the Hessian matrix, constructing W row-by-row, and construct the corresponding columns of ρᵤ′.

While the foregoing is a complete description of the embodiments of the invention, it should be evident that various modifications, alternatives and equivalents may be made and used. Accordingly, the above description should not be taken as limiting the scope of the invention which is defined by the metes and bounds of the appended claims.

What is claimed is:

1. A universally-applicable method of performing a multibody simulation, comprising
computing a derivative of a dynamic residual (DDR) with respect to a generalized coordinate in order (N²), and
using said derivative in said multibody simulation.

2. A method of claim 1, wherein said computing comprises
(i) breaking up the DDR into a first term and one or more additional terms, said first term containing a multibody operator and its transpose, and
(ii) evaluating said first term using a fast operator implementation.

3. A method of claim 2, wherein said DDR takes the form
$$\frac{\partial }{\partial q} {ρ}_{u} = H Φ P ( \frac{\partial }{\partial r} F ) {P}^{T} {Φ}^{T} {\tilde{H}}^{T} + \frac{\partial }{\partial q} ( H Φ P ) F .$$

4. A method of claim 2, wherein said evaluating comprises identifying a body Hessian (W) and reformulating said first term into an order (N²) expression.

5. A method of claim 4, wherein said evaluating comprises determining elements of said body Hessian locally.

6. A method of claim 5, wherein each of said elements is determined only once.

7. A method of claim 2, wherein said evaluating comprises defining an expression for omega (Ω).

8. A method of claim 7, wherein said expression for Ω takes the form Ω=ε_{φ}Ω+Ωε_{φ}^{T}−ε_{φ}Ωε_{φ}^{T}+W.

9. A method of claim 2, wherein said first term is used in the computation of a stiffness matrix.

10. A method of claim 2, wherein said first term is used in the computation of a Jacobian matrix

11. A method of claim 1, wherein said multibody simulation is a molecular simulation.

12. A method of claim 1, wherein said multibody simulation is performed using a computer.

13. A method of claim 1, wherein said generalized coordinate is a torsion angle coordinate.

14. A method of performing a simulation with a multibody system, comprising
evaluating
$$H Φ P \frac{\partial F }{\partial r} {P}^{T} {Φ}^{T} {\tilde{H}}^{T}$$
to calculate a torsion Jacobian matrix, and
using said torsion Jacobian matrix to determine characteristics of bodies of said multibody system in space.

15. A method of claim 14, wherein said characteristics include position and velocity of said bodies.

16. A method of claim 14, wherein said simulation is a molecular simulation.

17. A method of claim 14, wherein said using comprises defining an expression for omega (Ω) having the form Ω=ε_{φ}Ω+Ωε_{φ}^{T}−ε_{φ}Ωε_{φ}^{T}+W.

18. A method of determining characteristics of bodies in a multibody system, comprising
evaluating
$$H Φ P \frac{\partial F }{\partial r} {P}^{T} {Φ}^{T} {\tilde{H}}^{T}$$
to calculate a stiffness matrix, and
using said mass stiffness matrix to determine said characteristics.

19. A method of claim 18, wherein said multibody system represents one or more molecule(s) in a molecular simulation.

20. A method of claim 18, wherein said characteristics include normal modes of said system.
